(19) United States
(12) Patent Application Publication
(10) Pub. No.: US 2019/0328321 A1
(43) Pub. Date: Oct. 31, 2019

(54) SENSITIVITY METERING SYSTEM FOR USE IN PATIENT DIAGNOSIS FIELD

(71) Applicant: DYNAMIC DISC DESIGNS CORP.

(72) Inventor: Jerome Fryer, Nanaimo (CA)

(21) Appl. No.: 16/207,416

(22) Filed: Dec. 3, 2018

(30) Foreign Application Priority Data
Apr. 25, 2018 (CA) 3003155

(51) Int. Cl. A61B 5/00; A61B 5/22

(52) U.S. Cl. CPC A61B 5/4827; A61B 5/4836; A61B 5/0053; A61B 5/225

(57) ABSTRACT

A system to provide real-time feedback sensitivity of the patient's anatomical tissues while a practitioner probes with variations of pressure, whether in terms of force or direction, to determine the patient's sensitivity. The system may also be used to provide feedback when a patient undergoes movement such as active or passive ranges of motion. The system records a patient's sensitivity in a variable way as the practitioner provokes through movement of the anatomical tissues to help in the specificity of a diagnosis relating to tissue sensitivity.

### TECHNICAL FIELD

The present technology is directed to a compressible, deformable handheld device that allows a patient to report on relative sensitivity or discomfort, including methods, programs, and applications for use thereof.

### BACKGROUND

There are an ever-increasing number of therapies that involve assessing sensitivity, discomfort or mild pain through intense pain, for a patient. Practitioners of these therapies include, but are not limited to, chiropractors, physiotherapists and massage therapists, psychologists, osteopaths, naturopaths, dentists, medical doctors, orthopedists, etc. The patients can vary greatly in age, strength, cognitive ability and ability to communicate. It is known to be difficult to determine the level of sensitivity, discomfort or pain that a patient experiences, in real-time, without repeatedly asking the patient for feedback. Further, there are often no visual clues when the patient is experiencing sensitivity or mild discomfort. But even if there are indications, such as the patient's facial expressions, such visual indicators are not always visible and/or reliable.

### SUMMARY

The present technology is a feedback device and system, as well as methods, programs, and applications for use thereof, that allows a patient to report on levels of discomfort or sensitivity during treatment and/or a manual assessment as performed by chiropractors, physiotherapists and massage therapists, psychologists, osteopaths, naturopaths, dentists, medical doctors, orthopedists, etc. The system can be calibrated for each patient so as to pick up subtle differences particular to each patient during physical/mechanical diagnosis and treatment. A patient is also able to work within their desired compression range as the system can be calibrated for any range of forces. The system can track patient progress. The device may have a small form factor that is both light and easy for a patient to hold in their hand. The device may also have a surface that can be washed and disinfected.

The materials used in the device were selected to allow the patient to feel a change in shape of the device in response to the pressure exerted. This deformation of the device in response to the pressure exerted can transfer stress from the patient to the device in a measurable format.

The system can be used to provide real-time feedback sensitivity of the patient's anatomical tissues while a practitioner probes with variations of pressure and anatomical landmarks, whether in terms of force or direction, to determine the patient's sensitivity. The system may also be used to provide feedback when a patient undergoes movement like active or passive ranges of motion. The system records a patient's sensitivity in a variable way as the practitioner provokes through movement of the anatomical tissues to help in the specificity of a diagnosis relating to tissue sensitivity and/or real-time treatment.

In at least one embodiment, a patient specific sensitivity metering system for use in patient diagnosis and treatment includes a device that may be handheld, and that is deformable. The device includes a pressure sensor, a Bluetooth® radio and a battery; and the pressure sensor and the Bluetooth radio are both in electrical communication with the battery, while the pressure sensor is also in electrical communication with the Bluetooth radio. The system further includes a computing device that includes a Bluetooth® receiver, which is in radio communication with the Bluetooth radio, a processor and a memory that stores instructions thereon for calibrating the system to a specific patient. The memory also stores the calibration and sensitivity data set for the specific patient, as well as the ability to tag peaks of sensitivity with associated anatomy as identified by the practitioner. The system also includes a user interface, which is in electronic communication with the computing device.

In the sensitivity metering system, the handheld, deformable device may be portable, and may further comprise a wireless charger.

The sensitivity metering system may further comprise a doliometer, which may include a doliometer Bluetooth radio that is in radio communication with the Bluetooth receiver of the computing device to provide a doliometer data set to the memory.

In the sensitivity metering system, the memory may have instructions thereon to statistically analyze the sensitivity data set and the doliometer data set to provide a correlation value.

The computing device and the user display may be integrated into a handheld, mobile device.

The handheld mobile device may be a cell phone or a tablet.

The handheld, deformable device may include a skin and a body therein.

The body may be a silicone gel.

The handheld, deformable device may have a Shore OO rating between OO15 to OO40. Further, the device may be spherical in shape for handling.

The sensitivity metering system may further comprise one or more of a sound emitter or a patient user interface with a visual scale, the sound emitter and the patient user interface in electronic communication with the computing device.

In another embodiment, a method of assessing sensitivity of a selected body part of a patient to pressure or movement includes a practitioner selecting a sensitivity metering system, which includes a computing device and a deformable device. The deformable device includes a pressure sensor and an output that is in electronic communication with the pressure sensor and the computing device. The method further includes calibrating the sensitivity metering system for the patient to provide a patient specific calibration; storing the patient specific calibration in the system in the computing device; the practitioner exerting pressure or moving the selected body part; the patient squeezing the deformable device at a force commensurate with a perceived level of sensitivity; the pressure sensor sensing the force to provide a signal; the output sending the signal to the computing device; and the computing device analyzing the signal in relation to the patient specific calibration to provide a patient specific sensitivity data set.

The method may further include the practitioner selecting a doliometer and assessing an actual pressure exerted on the patient.

In yet another embodiment, a method of assessing sensitivity of a selected body part of a patient to pressure or movement includes a practitioner selecting a sensitivity metering system. The sensitivity metering system includes a handheld, deformable device which includes a pressure sensor, a Bluetooth® radio and a battery. The pressure sensor and the Bluetooth radio are in electrical communication with the battery, and the pressure sensor is in electrical communication with the Bluetooth radio. The sensitivity metering system also includes a computing device that has a Bluetooth® receiver, which is in radio communication with the Bluetooth radio, a processor and a memory. The memory stores instructions for calibrating the system to a specific patient, the calibration data, tagged peaks of sensitivity with associated anatomy as identified by the practitioner, and a sensitivity data set for the specific patient. The system further includes a user interface, which is in electronic communication with the computing device.

The method further includes calibrating the sensitivity metering system for the patient to provide a patient specific calibration; storing the patient specific calibration in the system in the computing device; the practitioner exerting pressure or moving the selected body part; the patient squeezing the handheld deformable device at a force commensurate with a perceived level of sensitivity; the pressure sensor sensing the force to provide a signal; the Bluetooth radio sending the signal to the computing device; and the computing device analyzing the signal to provide a patient specific sensitivity data set.

The method may further include the practitioner selecting a doliometer that includes a doliometer Bluetooth radio, which is in radio communication with the Bluetooth receiver of the computing device; the practitioner exerting pressure with the doliometer; the doliometer sensing the pressure to provide a doliometer signal; the doliometer Bluetooth radio sending the doliometer signal to the memory to provide a doliometer data set; the memory statistically analyzing the patient specific sensitivity data set and the doliometer data set to provide a correlation value; and the practitioner developing a treatment protocol.

### BRIEF DESCRIPTION OF THE DRAWINGS

In the detailed description that follows, embodiments are described as illustrations only since various changes and modifications will become apparent to those skilled in the art from the following detailed description. The use of the same reference numbers in different figures indicates similar or identical items.

FIG. 1 is a schematic of the system of the present technology.

FIG. 2 is a cross section of the deformable device of the system of FIG. 1.

FIG. 3 is a block diagram of the steps of the method of the present technology.

FIG. 4 is a schematic of an alternative embodiment of the system.

FIG. 5 is a block diagram of the steps of the method using the alternative embodiment.

FIG. 6 is a schematic of an alternative embodiment of the deformable device.

FIG. 7 is a schematic of another alternative embodiment of the deformable device.

### DETAILED DESCRIPTION

In the following detailed description, reference is made to the accompanying drawings, which form a part of the description. In the drawings, similar symbols typically identify similar components, unless context dictates otherwise. Furthermore, unless otherwise noted, the description of each successive drawing may reference features from one or more of the previous drawings to provide clearer context and a more substantive explanation of the current example embodiment. Still, the example embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein and illustrated in the drawings, may be arranged, substituted, combined, separated, and designed in a wide variety of different configurations, all of which are explicitly contemplated herein.

### Definitions

Computing device: in the context of the present technology, a computing device is any one of a cellular phone, a tablet, a laptop, desktop, or purpose-built computing device having a memory and a processor.

Handheld, mobile device: in the context of the present technology, a handheld, mobile device is a cell phone, a tablet, or a laptop.

Specific or selected parts of the body: in the context of the present technology, a specific or a selected part of the body is the part that a patient has concerns about, or is complaining about, or is a part of the body that the practitioner believes needs to be assessed in order to arrive at a diagnosis and treatment protocol.

Systems described herein may be calibrated for different patients and measuring techniques in order to obtain a meaningful assessment of levels of discomfort during manual diagnosis, range of motion assessments and treatment.

According to at least some embodiments, the systems allow a patient to report levels of discomfort or sensitivity during a manual assessment by, e.g., chiropractors, physiotherapists and massage therapists, psychologists, osteopaths, naturopaths, dentists, medical doctors, orthopedists, etc. The systems may be calibrated for each patient, thus facilitating detection of subtle differences for each individual during diagnosis and treatment.

Also, the systems facilitate tracking of a patient's progress during treatment. Thus, the form factor of at least one apparatus corresponding to the system may be held in a patient's hand. Therefore, for an evaluation and/or treatment environment, the apparatus may be made of materials that are easily washed and disinfected. As a hand-held device, the tactile feel of the apparatus may change in response to the pressure exerted by the patient to provide a means for stress transference for the patient.

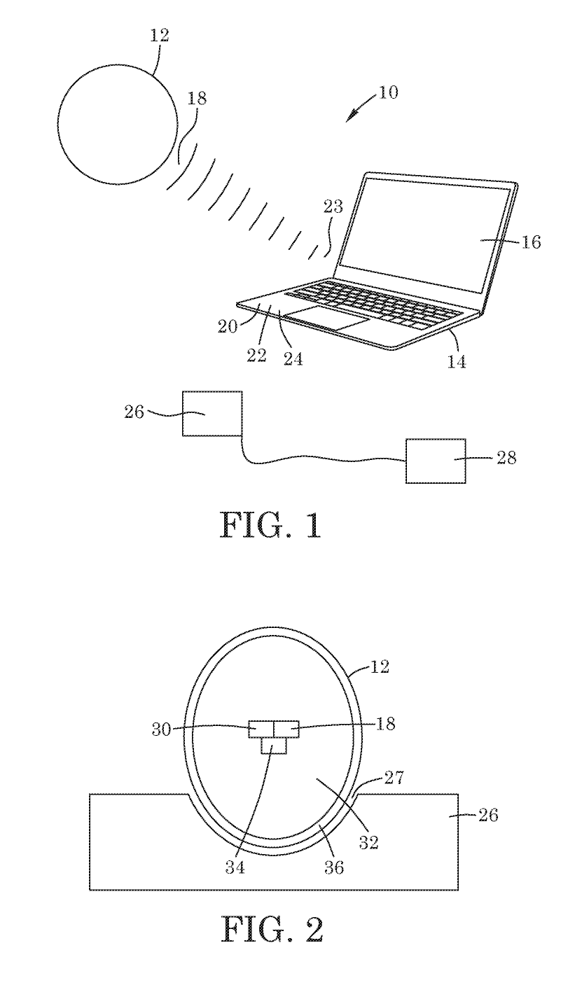

The system also facilitates real-time feedback regarding the sensitivity of the patient's anatomical tissues while undergoing medical treatment, e.g., when a practitioner probes portions of the patient's anatomy with variations of pressure and anatomical landmarks to determine the patient's sensitivity. Also, the system facilitates feedback from a patient whose anatomy undergoes testing for range of motion. Therefore, the system records a patient's sensitivity in a variable way as a practitioner provokes movement of anatomical tissues to help in the specificity of a diagnosis relating to tissue sensitivity. A sensitivity metering system 10 is shown in FIG. 1. It includes a compressible, deformable device 12, which may be handheld in accordance with some embodiments, but may also be compressed with a foot, or other body part as needed; a computing device 14; and a display or user interface 16. The compressible device 12 communicates via a Bluetooth® radio 18 to the computing device 14, which has a processor 20 to receive instructions from a memory 22, and a Bluetooth receiver 23. The processor 20, under control of the memory 22, converts the pressure information into data, which is then stored in the memory 22. The memory 22 includes an application 24 that calibrates the force, on a percentage scale, exerted by the patient as (s)he squeezes the device 12. The computing device 14 is in electronic communication with the user interface 16, which may be integral with the computing device 140 or may be separate to the computing device 14. A wireless charger 26 is in wireless communication with the system to charge the system. The wireless charger 26 is in electrical communication with the computing device 14 or another power source 28.

As shown in FIG. 2, the wireless charger 26 has a concavity 27 in which the handheld device 12 can rest during charging or when not in use. The compressible handheld device 12 may be spherical or egg-shaped, and therefore is portable. A pressure sensor 30 is located within the body 32 of the device. The preferred pressure sensor is a piezo-electric sensor or a micro-electromechanical (MEMS) pressure sensor connected to the Bluetooth radio 18. A battery 34 is also housed in the body 32 of the device 12. The electronics of the device (the pressure sensor 30, the Bluetooth radio 18 and the battery 34) are of such form factor so as not to interfere with deformation of the handheld device 12 in response to pressure. The skin 36 of the device may be washable and, therefore may be disinfected, which is preferable in an environment for evaluation and/or treatment. The skin 36 and body 32 of the device may change shape in response to pressure exerted on the device 12. The skin 36 is a flexible plastic polymer. The body 32 is preferably a silicone gel.

For a healthy adult having an average grip strength of, e.g., 86 Newtons/centimeter² for a grip by which the whole hand closes on a dynamometer (referred to as a grip) down to as low as 13 Newtons/centimeter² for a tip pinch, or for a healthy child having an average grip strength from about 0 Newtons/centimeter² to about 7 Newtons/centimeter² for a tip pinch, materials with different durometer ratings were considered and tested for the device. An elastomer with a Shore OO durometer rating of about OO15 to about OO40, preferably about OO20 to about OO30 provides sufficient resiliency to protect the electronics in the body 32 of the device 12, while providing immediate tactile feedback to the patient in terms of deformation of the device 12. Device 12 may be produced to fit children or adults. One of these two sizes were found to be comfortably held and squeezed by a wide range of patients.

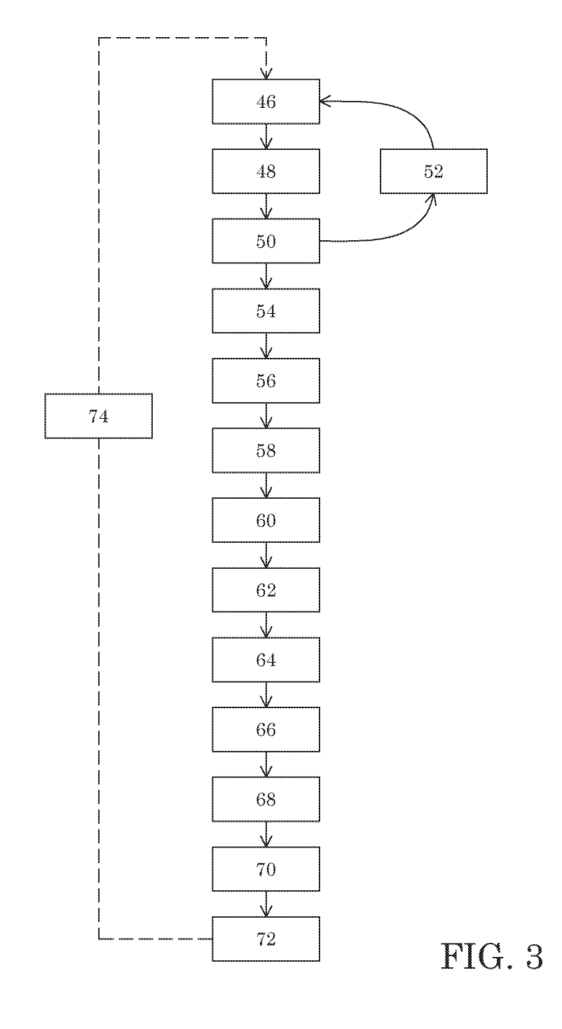

As shown in FIG. 3, prior to assessment, the patient holds 46 the device 12 with what the patient perceives as no pressure. The patient may be asked to squeeze 48 the device as hard as possible. That effort at squeezing may be calculated as 100%, and the range and sensitivity of measurement for device 12 may be adjusted accordingly. If 100% is 7, then data collection will be based, for example, on increments of 0.1. If 100% is 70, then data collection will be based on increments of 1.0. This calibration controls the noise in the system for those patients exerting higher pressure, while keeping the system sensitive enough for the practitioner to see a range of responses in the weaker patients. The data from these two pressures (no pressure and full pressure) may then be converted 50 to a percentage, with no pressure being 0% and full pressure being 100%. The patient repeats 52 this at least three times. The application 40 calculates 54 the mean and stores 56 the calibration in the memory 22 in association with a patient identifier. The practitioner may then input 58 the patient identifier and begin an assessment 60 by one or more of palpating, exerting gentle pressure on a specific part of the patient's body, gently manipulating the patient's body or having the patient move through a range of motion. The specific or selected parts of the body are the parts of the body needing a diagnosis or are parts of the body that the practitioner believes need to be assessed in relation to the patient's concerns or complaints. The patient squeezes 62 the compressible handheld device 12 in response to physical stimuli. The pressure sensor registers 64 the pressure and an electrical output representative of the force of the pressure is sent 66 to the Bluetooth radio 18, which wirelessly transmits 68 the raw data to the application 24, where it is processed 70 using the previously stored calibration and/or tags for peaks of sensitivity for the specific patient. The data may then be stored 72 for use in tracking how the patient is responding to treatment. In this manner, an accurate assessment of the patient's areas of sensitivity or discomfort are identified, without the health care provider having to induce pain in order to assess the patient's condition. In patients that are clearly becoming stronger or weaker, calibration can be repeated 74 as needed. Further, the calibration can be done for either a pinch, such as a tip pinch, or a grip, noting that the force exerted in these different holds are very different.

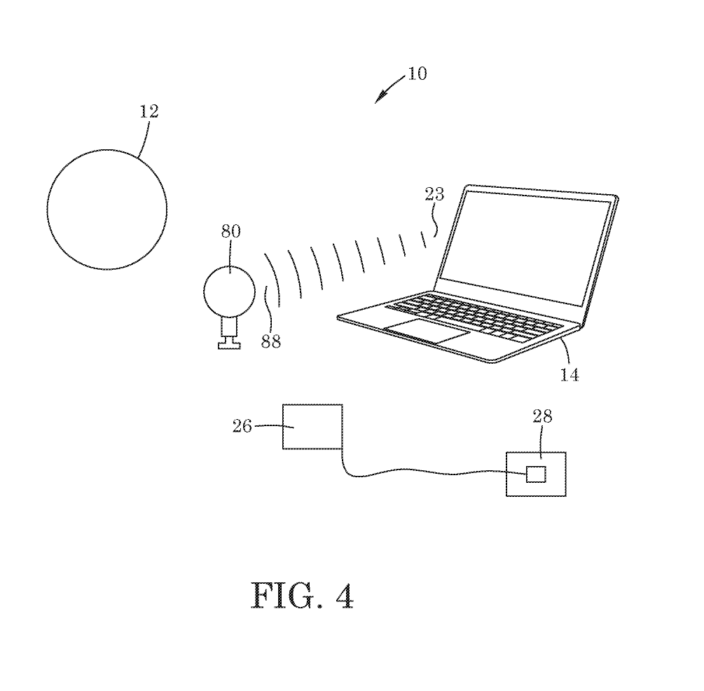

As shown in FIG. 4, the sensitivity system can further include a doliometer 80, which reports on the actual pressure being exerted by the practitioner on the patient. The doliometer 80 communicates via a Bluetooth® radio 88 to the computing device 14. The processor 20, under control of the memory 22, statistically analyzes the correlation between the doliometer reading and the patient's response, in terms of compression of the device 12, expressed in percentage for that patient. The results are stored in the memory 22.

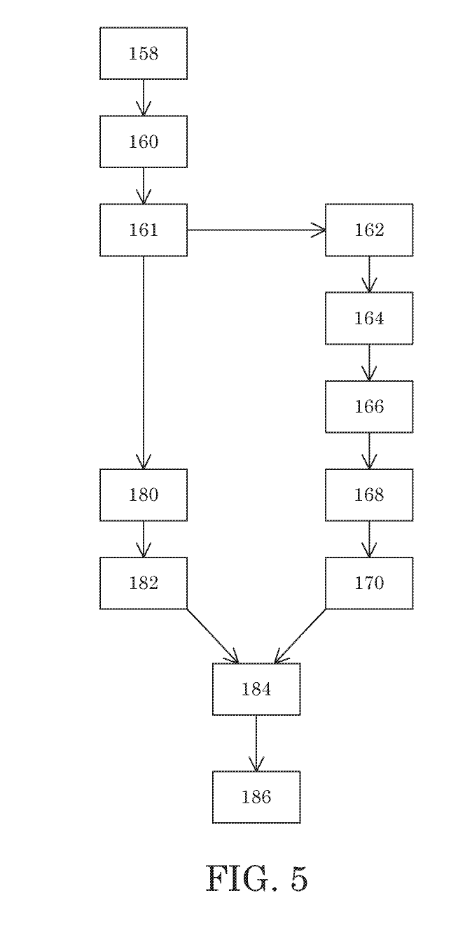

A block diagram of the steps when a doliometer is included in the system is shown in FIG. 5. The system is calibrated as described and shown in FIG. 3. The practitioner inputs 158 the patient identifier, and begins an assessment 160 by one or more of palpating, exerting gentle pressure on specific parts of the patient's body, gently manipulating the patient's body or having the patient move through a range of motion. The specific or selected parts of the body are the parts of the body needing a diagnosis or are parts of the body that the practitioner believes need to be assessed in relation to the patient's concerns or complaints. The practitioner uses 161 the doliometer 80 when palpating or exerting pressure on the patient. Concomitantly, the patient squeezes 162 the compressible handheld device 12 in response to the sensation that they feel. The pressure sensor registers 164 the pressure, and an electrical output representative of the force of the pressure is sent to 166 the Bluetooth radio 18, which wirelessly transmits 168 the raw data to the application 24, where it is processed 170 using the previously stored calibration and/or tags for peaks of sensitivity for the specific patient. The doliometer 80 registers 180 the actual pressure exerted, and an electrical output representative of the force of the pressure is sent to the Bluetooth radio 88, which wirelessly transmits 182 the raw data to the application 24. The application statistically analyzes 184 the correlation between the doliometer reading and the patient's response, in terms of compression of the device 12, expressed in percentage for that patient. The results are stored in the memory 186. In this manner, an accurate assessment of the patient's areas of sensitivity or discomfort are identified, without the health care provider having to induce pain in order to assess the patient's condition. In patients that are clearly becoming stronger or weaker, calibration may be repeated 188 as needed. Further, the calibration can be done for either a pinch, such as a tip pinch, or a grip, noting that the force exerted in these different holds are very different.

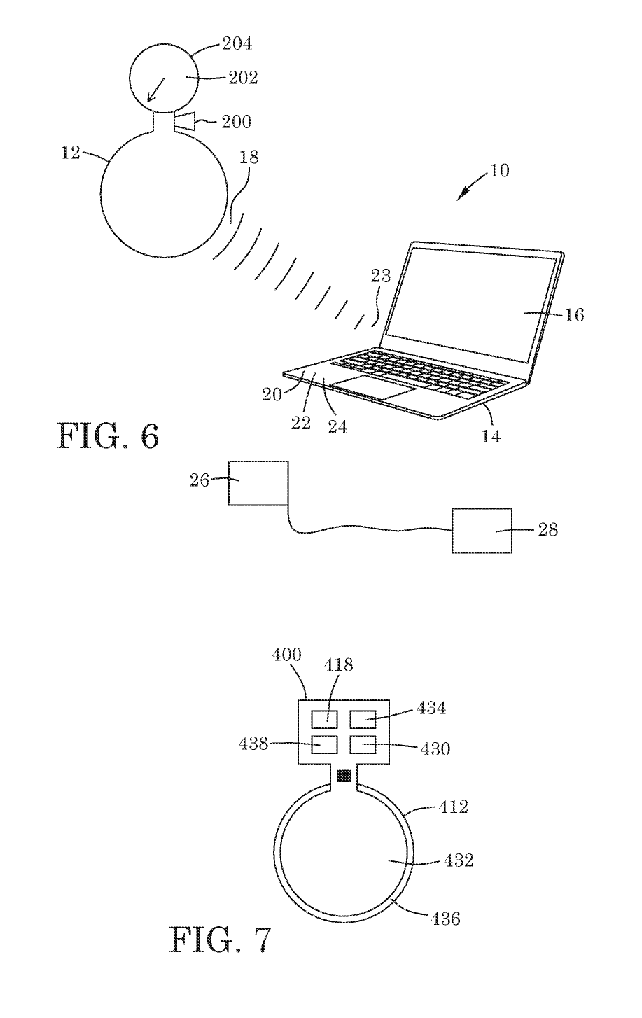

In another embodiment shown in FIG. 6, the system 10 has sound emitter 200 that emits an audible signal. The signal is emitted at the pressure corresponding to about 95% to about 100% pressure for that patient. The system 10 may also include a visual display 202 on a patient user interface 204. The visual display 202 is a dial or bar that shows the patient's feedback in terms of the percentage of pressure for that patient. The sound emitter 200 and the patient user interface 204 are in electronic communication with the computing device 14.

In another embodiment shown in FIG. 7, the pressure sensor 430 is in fluid communication with the body 432 of the device 412. The preferred pressure sensor is a piezo-electric sensor connected to the Bluetooth radio 418. The electronics of the device (the pressure sensor 430, the Bluetooth radio 418 and the battery 434) are retained on a board 400, which is attached to the skin 436. The board 400 may include an Arduino board 438.

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims.

We claim:

1. A patient-specific sensitivity metering system for use in patient diagnosis and treatment, the sensitivity metering system comprising:
a handheld device, including:
a pressure sensor,
a Bluetooth® radio, and
a battery,
wherein the pressure sensor and the Bluetooth radio are in electrical communication with the battery, and the pressure sensor is in electrical communication with the Bluetooth radio;
a computing device, including:
a Bluetooth® receiver, which is in radio communication with the Bluetooth radio,
a processor,
a memory storing instructions thereon for calibrating the system to a specific patient, the calibration data, and sensitivity data set for the specific patient, and
a user interface, which is in electronic communication with the computing device.

2. The sensitivity metering system of claim 1, wherein the handheld device is portable.

3. The sensitivity metering system of claim 1, further comprising a wireless charger.

4. The sensitivity metering system of claim 1, further comprising a doliometer.

5. The sensitivity metering system of claim 4, wherein the doliometer includes a doliometer Bluetooth radio, and wherein the doliometer Bluetooth radio is in radio communication with the Bluetooth receiver of the computing device to provide a doliometer data set to the memory.

6. The sensitivity metering system of claim 5, the memory storing instructions thereon to statistically analyze the sensitivity data set and the doliometer data set to provide a correlation value.

7. The sensitivity metering system of claim 1, wherein the computing device and the user display are integrated into a handheld, mobile device.

8. The sensitivity metering system of claim 7, wherein the handheld mobile device is a cell phone or a tablet.

9. The sensitivity metering system of claim 1, wherein the handheld device includes a skin and a body therein.

10. The sensitivity metering system of claim 9, wherein the body is a silicone gel.

11. The sensitivity metering system of claim 1, wherein the handheld device has a Shore 00 rating between 0015 to 0040.

12. The sensitivity metering system of claim 1, further comprising one or more of a sound emitter or a patient user interface with a visual scale, wherein the sound emitter and the patient user interface are in electronic communication with the computing device.

13. The sensitivity metering system of claim 1, wherein the handheld device is spherical.

14. A method of assessing sensitivity of a selected body part of a patient to pressure or movement using a sensitivity metering system that includes a computing device and a portable device that has a pressure sensor and an output, with the output being in electronic communication with the pressure sensor and the computing device, the method comprising:
calibrating the sensitivity metering system for the patient to provide a patient specific calibration;
storing the patient specific calibration in the system in the computing device;
the pressure sensor of the portable device detecting pressure from the patient at a force commensurate with a perceived level of sensitivity in response to a body part moving or having pressure applied thereto, to provide a signal;
the output sending the signal to the computing device; and
the computing device analyzing the signal in relation to the patient specific calibration to provide a patient specific sensitivity data set.

15. The method of claim 14, wherein the sensitivity metering system comprises:
a handheld device, including:
a pressure sensor,
a Bluetooth® radio, and
a battery,
wherein the pressure sensor and the Bluetooth radio are in electrical communication with the battery, and the pressure sensor is in electrical communication with the Bluetooth radio;
a computing device including a Bluetooth® receiver, which is in radio communication with the Bluetooth radio,
a processor;
a memory storing instructions thereon for calibrating the system to a specific patient, the calibration data, and sensitivity data set for the specific patient; and
a user interface, which is in electronic communication with the computing device

16. The method of claim 14, further comprising:
a doliometer included in the system sensing the pressure exerted by a practitioner to provide a doliometer signal, and
a doliometer Bluetooth radio sending the doliometer signal to the memory to provide a doliometer data set.

17. The method of claim 16, further comprising:
the memory statistically analyzing the patient specific sensitivity data set; and
the doliometer data set to provide a correlation value.

19. A method of assessing sensitivity of a selected body part of a patient to pressure or movement by a computing device, the method comprising:
calibrating a handheld device, via a Bluetooth connection, in order for a patient to provide a patient-specific calibration, using instructions stored in a memory on the computing device;
receiving, from the handheld device, a signal commensurate with a force by which the patient exerted pressure on the handheld device; and
analyzing the signal to provide a patient specific sensitivity data set.

20. The method of claim 19, further comprising:
receiving, via another Bluetooth connection, a doliometer signal indicative of pressure exerted by the patient on a doliometer; and
computing a patient-specific doliometer data set.

21. The method of claim 20, wherein the computing comprises:
statistically analyzing the patient-specific sensitivity data set; and
deriving a correlation value between the patient-specific sensitivity data set and the doliometer data set.
